(19) United States
(12) Patent Application Publication
(10) Pub. No.: US 2026/0325054 A1
(43) Pub. Date: Oct. 22, 2026

(54) CLOCK DRAWING TEST EVALUATION APPARATUS AND METHOD

(71) Applicant: Electronics and Telecommunications Research Institute, Daejeon (KR)

(72) Inventors: Min-Ho BAE, Seongnam-si (KR); Seong-Hee LEE, Gwangju-si (KR)

(73) Assignee: Electronics and Telecommunications Research Institute, Daejeon (KR)

(21) Appl. No.: 19/537,791

(22) Filed: Feb. 12, 2026

(30) Foreign Application Priority Data
Apr. 17, 2025 (KR) 10-2025-0050379

(51) Int. Cl. A61B 5/00; A61B 5/11; G06T 7/00; G06T 11/23

(52) U.S. Cl. CPC A61B 5/4088; A61B 5/1124; A61B 5/7246; A61B 5/7264; G06T 7/0014; G06T 11/23; G06T 2207/10024; G06T 2207/20081; G06T 2210/41

(57) ABSTRACT

Disclosed herein are a clock drawing test evaluation apparatus and method. The clock drawing test evaluation apparatus includes a processor, and a memory configured to store an instruction that is executed by the processor, wherein the processor is configured to convert sketch data of a target clock image that is a subject of clock drawing test into a stroke-unit image including time information, extract feature vectors of the stroke-unit image from the stroke-unit image through a learning model, and then evaluate the clock drawing test based on the feature vectors of the stroke-unit image.

### CROSS REFERENCE TO RELATED APPLICATION

This application claims the benefit of Korean Patent Application No. 10-2025-0050379, filed Apr. 17, 2025, which is hereby incorporated by reference in its entirety into this application.

### BACKGROUND OF THE INVENTION

### 1. Technical Field

The present disclosure relates to a clock drawing test evaluation apparatus and method.

### 2. Description of the Related Art

Dementia is one of the greatest medical and social issues in today's rapidly aging society, and is regarded as one of the diseases most feared by middle-aged and older adults. Dementia leads to a loss of self, is accompanied by changes in social roles and increased dependency, and imposes substantial economic and emotional burdens not only on individuals but also on their families and society. Moreover, as life expectancy increases, the incidence of neurodegenerative diseases such as dementia is rising, and thus methods for preventing and enabling early diagnosis of such diseases have emerged as a critical issue.

As one of early diagnostic methods for dementia, a Clock Drawing Test (CDT) is widely utilized. The clock drawing test is a useful tool that enables simple and rapid assessment of cognitive function through the process in which a user correctly places the time and numerals on a clock. However, scoring of the clock drawing test relies heavily on the subjective judgment of specialists, which limits the consistency and objectivity of the results. Further, efficiency issues may arise when large-scale tests are conducted.

The background technology of the present disclosure is disclosed in Korean Patent No. 10-2554315.

### SUMMARY OF THE INVENTION

Accordingly, the present disclosure has been made keeping in mind the above problems occurring in the prior art, and an object of the present disclosure is to provide a clock drawing test evaluation apparatus and method, which are capable of rapidly and consistently evaluating whether a user exhibits a decline in cognitive function by analyzing a clock image drawn by the user in real time.

In accordance with an aspect of the present disclosure to accomplish the above object, there is provided a clock drawing test evaluation apparatus, including a processor, and memory configured to store an instruction that is executed by the processor, wherein the processor is configured to convert sketch data of a target clock image that is a subject of clock drawing test into a stroke-unit image including time information, extract feature vectors of the stroke-unit image from the stroke-unit image through a learning model, and then evaluate the clock drawing test based on the feature vectors of the stroke-unit image.

The processor may be configured to assign a stroke order and range, a drawing time of the clock image, and a time required to draw each stroke to three RGB channels, respectively.

The processor may be configured to designate a color according to an order of strokes and normalize a range of strokes to assign a normalized stroke range to an R channel, normalize the drawing time of the clock image to assign the normalized drawing time to a G channel, normalize a ratio of a time required to draw each stroke to a time required to complete an entire clock image to assign the normalized ratio to a B channel.

The learning model may extract the feature vectors of the stroke-unit image and learn a distance proportional to a difference between scores of respective clock images based on the feature vectors of the stroke-unit image.

The learning model may learn the feature vectors of the stroke-unit image through a Euclidean-distance-based loss function, and the loss function may reflect a distance margin in proportion to the difference between the scores of the clock images.

The processor may be configured to extract a score of the target clock image through a comparison of a similarity between feature vectors of the stroke-unit image and feature vectors previously stored in a database unit.

The processor may be configured to determine a score of any one feature vector having a highest similarity to the feature vectors of the stroke-unit image, among the feature vectors stored in the database unit, to be the score of the target clock image.

In accordance with an aspect of the present disclosure to accomplish the above object, there is provided a clock drawing test evaluation method, including converting, by a processor, sketch data of a target clock image that is a subject of clock drawing test into a stroke-unit image including time information, and extracting, by a processor, feature vectors of the stroke-unit image from the stroke-unit image through a learning model, and then evaluating the clock drawing test based on the feature vectors of the stroke-unit image.

In converting into the stroke-unit image, the processor may be configured to assign a stroke order and range, a drawing time of the clock image, and a time required to draw each stroke to three RGB channels, respectively.

In converting into the stroke-unit image, the processor may be configured to designate a color according to an order of strokes and normalize a range of strokes to assign a normalized stroke range to an R channel, normalize the drawing time of the clock image to assign the normalized drawing time to a G channel, normalize a ratio of a time required to draw each stroke to a time required to complete an entire clock image to assign the normalized ratio to a B channel.

The learning model may extract feature vectors of the stroke-unit image and learn a distance proportional to a difference between scores of respective clock images based on the feature vectors of the stroke-unit image.

The learning model learns the feature vectors of the stroke-unit image through a Euclidean-distance-based loss function, and the loss function may reflect a distance margin in proportion to the difference between the scores of the clock images.

In evaluating the clock drawing test, the processor may be configured to extract a score of the target clock image through a comparison of a similarity between feature vectors of the stroke-unit image and feature vectors previously stored in a database unit.

In evaluating the clock drawing test, the processor may be configured to determine a score of any one feature vector having a highest similarity to the feature vectors of the stroke-unit image, among the feature vectors stored in the database unit, to be the score of the target clock image.

### BRIEF DESCRIPTION OF THE DRAWINGS

The above and other objects, features and advantages of the present disclosure will be more clearly understood from the following detailed description taken in conjunction with the accompanying drawings, in which:

FIG. 1 is a block configuration diagram of a clock drawing test evaluation apparatus according to an embodiment of the present disclosure;

FIG. 2 is a block configuration diagram of a processor according to an embodiment of the present disclosure;

FIG. 3 is a diagram illustrating a training method of a learning model according to an embodiment of the present disclosure;

FIG. 4 is a diagram illustrating an evaluation method of an evaluation unit according to an embodiment of the present disclosure; and

FIG. 5 is a flowchart of a clock drawing test evaluation method according to an embodiment of the present disclosure.

### DESCRIPTION OF THE PREFERRED EMBODIMENTS

Hereinafter, embodiments of a clock drawing test evaluation apparatus and method according to embodiments of the present disclosure will be described. In this process, the thickness of lines or the sizes of components illustrated in the drawings may be exaggerated for clarity and convenience of description. The terms, which will be described later, may be defined in consideration of the functions in the present disclosure, and may be changed depending on the intention or custom of a user or an operator. Therefore, these terms should be defined based on the entire contents of the present specification.

The present disclosure may be implemented in different forms, and is not limited to embodiments described here. In the drawings, portions irrelevant to the description are omitted to clearly illustrate the present disclosure, and similar reference numerals are used to designate similar parts throughout the specification.

In the specification, when a certain portion is referred to as “comprising” or “including” a component, it does not preclude another component but may further include other components unless the context clearly indicates otherwise.

Implementations described in the present specification may be embodied, for example, in a method or process, an apparatus, a software program, a data stream, or a signal. Even if discussed only in the context of a single form of implementation (e.g., only as a method), the features discussed may also be implemented in other forms (e.g., as an apparatus or program). The apparatus may be implemented in suitable hardware, software, firmware, or the like. The method may be implemented in an apparatus such as a processor, which generally refers to a processing device including, for example, a computer, a microprocessor, an integrated circuit, or a programmable logic device.

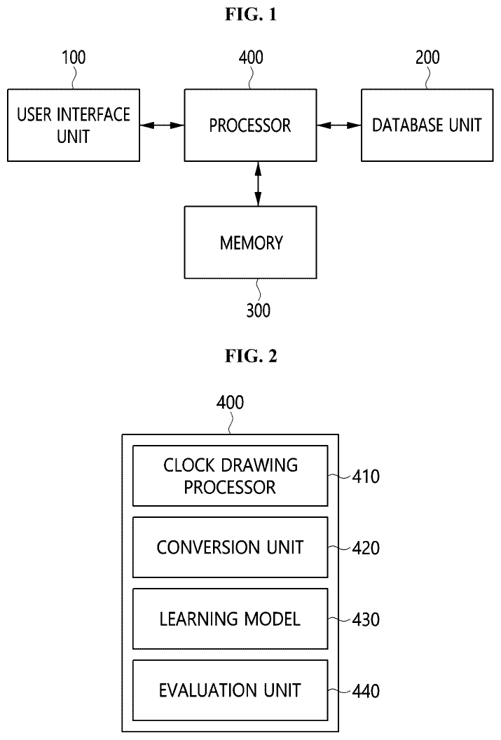

FIG. 1 is a block configuration diagram of a clock drawing test evaluation apparatus according to an embodiment of the present disclosure.

Referring to FIG. 1, the clock drawing test evaluation apparatus according to the embodiment of the present disclosure may include a user interface 100, a database (DB) unit 200, memory 300, and a processor 400.

In the present embodiment, clock images are categorized into and described as a target clock image, which is drawn by a user and serves as the subject of the Clock Drawing Test; and a training clock image, which serves as training data for a learning model.

The user interface unit 100 may provide a user interface. For example, the user interface unit 100 may provide various user interfaces allowing the user to draw the target clock image. The user interface unit 100 may output the result of evaluation of a Clock Drawing Test (CDT). The evaluation result may be a score for the target clock image based on the clock drawing test evaluation, or may indicate whether the user exhibits a decline in cognitive function, but the evaluation result is not especially limited thereto.

The user interface unit 100 may include, for example, a keyboard, a mouse, a touchpad, a touch screen, an electronic pen, touch buttons, and the like. The user interface unit 100 may include a display or the like to output data. The display may be implemented as, for example, a thin film transistor-liquid crystal display (TFT-LCD) panel, a light emitting diode (LED) panel, an organic LED (OLED) panel, an active matrix OLED (AMOLED) panel, a flexible panel, or the like.

In an embodiment, the above-described user interface unit 100, database unit 200, memory 300, and processor 400 may be mounted to be integrated into an electronic device (not illustrated). The electronic device may be, but is not limited to, a mobile phone, a smart phone, a notebook computer, a tablet computer, or a desktop computer.

In another embodiment, the user interface unit 100 may be mounted on an electronic device with which a target clock image can be drawn, and the database unit 200, the memory 300, and the processor 400 may be mounted on a separate server (not illustrated) and connected to the electronic device via a communication network. The communication network may employ 3rd Generation Partnership Project (3GPP), Long Term Evolution (LTE), 5th Generation (5G), Worldwide Interoperability for Microwave Access (WiMAX), wired and wireless Internet, a Local Area Network (LAN), a Wireless Local Area Network (wireless LAN), a Wide Area Network (WAN), a Personal Area Network (PAN), Bluetooth, Wireless Fidelity (Wi-Fi), and the like, but is not limited thereto. In this case, the electronic device may generate a target clock image in response to a control command that is input through the user interface unit 100, and may deliver the generated target clock image to the processor 400 over the communication network, thus allowing the processor 400 to evaluate the clock drawing test.

The database unit 200 may store feature vectors for extracting the score of the target clock image. The feature vectors may be generated in advance from training clock images through the learning model, and may be stored. The feature vectors will be described later. Further, the database unit 200 may store a feature vector generated from the target clock image.

The memory 300 may store various types of data used by the processor 400. The data may include instructions allowing operations or steps according to an embodiment of the present disclosure to be performed. That is, the memory 300 may store instructions that enable the execution of a clock drawing test designed for dementia prevention using artificial intelligence models.

The memory 300 may include a storage medium corresponding to at least one of a flash memory type, a hard disk type, a multimedia card micro type (MMC micro), a card-type memory, a random access memory (RAM), a static random access memory (SRAM), a read-only memory (ROM), a programmable read-only memory (PROM), an erasable programmable read-only memory (EPROM), or an electrically erasable programmable read-only memory (EEPROM), or a combination thereof.

Furthermore, the processor 400 may be configured such that components for performing respective functions are classified at hardware, software, or logic levels. In this case, dedicated hardware for performing each function may be used. For this, the processor 400 may be implemented as, or include, at least one of an Application Specific Integrated Circuit (ASIC), a Digital Signal Processor (DSP), Programmable Logic Devices (PLD), Field Programmable Gate Arrays (FPGAs), a Central Processing Unit (CPU), a microcontroller, and/or a microprocessor, or a combination thereof.

The processor 400 may be connected to the memory 300, and may execute instructions stored in the memory 300. The processor 400 may execute the instructions stored in the memory 300 to control at least one other component (e.g., hardware or software component) connected to the processor 400, and may perform various data processing or computation operations.

The processor 400 may be implemented as a Central Processing Unit (CPU) or a System on Chip (SoC), may control multiple hardware or software components connected to the processor 400 by running an operating system or an application, and may perform various types of data processing and computation operations. The processor 400 may execute at least one instruction stored in the memory 300, and may store data indicating the result of execution in the memory 300.

The processor 400 may evaluate the degree of dementia using an artificial intelligence model based on the target clock image drawn by the user. In this case, the processor 400 may convert sketch data of the target clock image into stroke-unit images including time information. The processor 400 may extract feature vectors from the stroke-unit images through the learning model. The processor 400 may extract a score of the target clock image by comparing similarities between the feature vectors of the stroke-unit images and feature vectors previously stored in the database unit 200. The score of the target clock image may serve as a criterion for determining whether the user exhibits a decline in cognitive function.

FIG. 2 is a block configuration diagram of a processor according to an embodiment of the present disclosure.

Referring to FIG. 2, the processor 400 may include a clock drawing processor 410, a conversion unit 420, a learning model 430, and an evaluation unit 440.

The clock drawing processor 410 may generate a target clock image in response to a control command of the user, received from the user interface unit 100.

When the clock drawing test is evaluated, the conversion unit 420 may convert vector-format sketch data of the target clock image into stroke-unit images including time information. Various types of information may be reflected in the stroke-unit images. That is, the processor 400 may assign a stroke order and range, a drawing time of the target clock image, and the time required to draw each stroke to RGB three channels (8 bits) of the stroke-unit images.

The conversion unit 420 may designate the color of R channel (Red) according to the order of strokes, set the stroke range from a minimum value of 1 to a maximum value of 50, normalize the stroke range to a range of 0 to 255, and assign normalized values to the R channel. Here, the reason for setting the minimum value to 1 is that a case in which no stroke is present is not regarded as a normal clock image. The reason for setting the maximum value to 50 is that the maximum number of strokes in a clock image is generally about 50.

The conversion unit 420 may normalize the drawing time of the target clock image to the range of 0 to 255, and may assign the normalized value to a Green (G) channel.

To reflect the time required to draw a specific stroke, the conversion unit 420 may calculate a ratio between the time required to draw the corresponding stroke on a stroke basis and the time required to complete the target clock image, normalize the calculated ratio to the range of 0 to 255, and reflect the normalized ratio in a Blue (B) channel.

The conversion unit 420 may convert vector-format sketch data of the training clock image into stroke-unit images including time information.

During the training of the learning model 430, the conversion unit 420 may convert the vector-format sketch data of the training clock image into stroke-unit images including time information.

The learning model 430 may learn a distance proportional to the score of the training clock image based on the feature vectors of the stroke-unit images converted from the training target image. That is, the learning model 430 may perform learning based on distances proportional to the differences between scores of the training clock images by reflecting the scores of respective training clock images in a training process. For example, based on the Shulman score (with a maximum of 6 points), which is commonly used in clock drawing test evaluation, training clock images with scores of 5 and 4 are structurally more similar to each other, whereas training clock images with scores of 5 and 1 exhibit a large structural difference. The learning model 430 represents the similarities between training clock images as distances by reflecting the above-described scores of the training clock images in training, thus enabling training clock images with higher scores to share more similar features and enabling training clock images with lower scores to have larger differences.

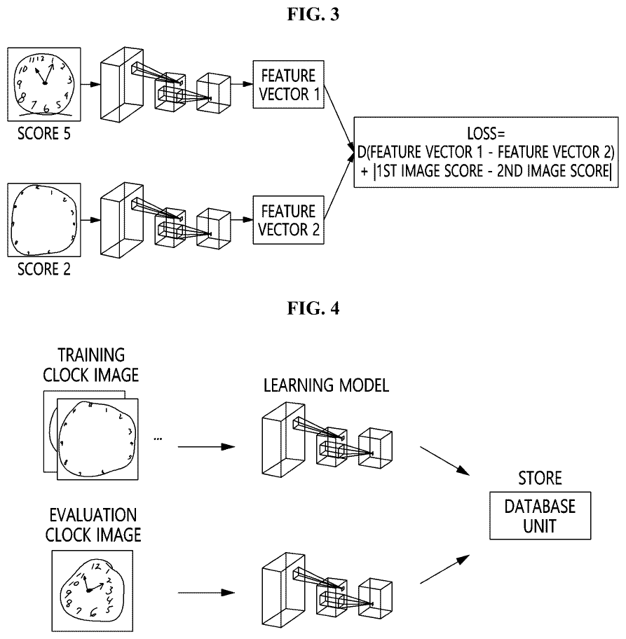

FIG. 3 is a diagram illustrating a training method of a learning model according to an embodiment of the present disclosure.

In FIG. 3, a method for extracting feature vectors from generated target clock images and learning distances proportional to the scores of training clock images based on the extracted feature vectors is illustrated.

During a process of extracting feature vectors of training clock images, a Convolutional Neural Network (CNN) may be used. When the feature vectors of the training clock images are extracted through a feature extraction module of the CNN, the extracted feature vectors may be learned through a Euclidean distance-based loss function, and may be designed such that a distance margin is reflected in proportion to the difference between the scores of the training clock images. In detail, the following loss function may be defined such that a pair of training clock images with a larger score difference is trained with a larger distance margin, and a pair of training clock images with a smaller score difference is trained with a smaller distance margin.

loss=Distance(1st image feature vector−2nd image feature vector)+|1st image score−2nd image score|

The loss function may be a method for setting distance in proportion to the difference between the scores of the training clock images, and may train similarity and differentiation between the training clock images. This reflects that, in clock drawing test evaluation, training clock images with high scores share similar structural features, whereas training clock images with low scores have different structural features. Therefore, the learning model 430 may clearly identify the distance between training clock images using the difference between the scores of the training clock images, and may learn image features depending on the scores of the training clock images, thus enabling highly reliable clock drawing test evaluation.

During training of the learning model, the evaluation unit 440 may extract the feature vectors of the training clock images through the learning model 430 and may evaluate the extracted feature vectors. In this case, the evaluation unit 440 may store the feature vectors of all training clock images used for training in the database unit 200.

During evaluation of a clock drawing test, the evaluation unit 440 may extract the feature vectors of the target clock images through the learning model 430. The evaluation unit 440 may search, among the feature vectors stored in the database unit 200, for a feature vector most similar to a feature vector of a corresponding target clock image, and may determine a score corresponding to the found feature vector to be the score of the corresponding target clock image.

FIG. 4 is a diagram illustrating an evaluation method of an evaluation unit according to an embodiment of the present disclosure.

Referring to FIG. 4, an example is illustrated in which the evaluation unit 440 stores all feature vectors of training clock images used for training in the database unit 200, and searches the database unit 200 for a feature vector that is most similar to a feature vector of a corresponding target clock image among the feature vectors stored in the database unit 200.

In the present embodiment, for better understanding of the embodiment, the clock drawing processor 410, the conversion unit 420, the learning model 430, and the evaluation unit 440 are described as separate components in the processor 400, but, in other embodiments, the processor 400 may be implemented as a configuration for performing functions of individual sub-components in an integrated manner.

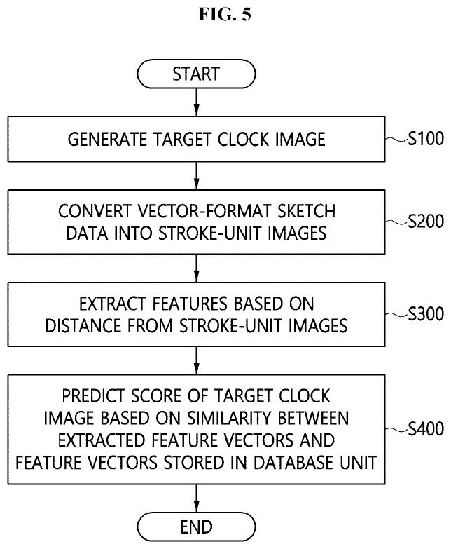

FIG. 5 is a flowchart of a clock drawing test evaluation method according to an embodiment of the present disclosure.

Referring to FIG. 5, the clock drawing processor 410 may generate a target clock image in response to a control command of a user, received from the user interface unit 100 at step S100.

The conversion unit 420 may convert vector-format sketch data of the target clock image into stroke-unit images including time information at step S200. In this case, the processor 400 may assign an order of strokes, a drawing time of the target clock image, and the time required to draw each stroke to RGB three channels (8 bits).

The evaluation unit 440 may extract feature vectors of the target clock image through the learning model 430 at step S300.

The evaluation unit 440 may predict a score of the target clock image by analyzing similarities between the feature vectors of the target clock image and feature vectors previously stored in the database unit 200 at step S400. That is, the evaluation unit 440 may search, among the feature vectors stored in the database unit 200, for a feature vector most similar to a feature vector of a corresponding target clock image, and may determine a score corresponding to the found feature vector to be the score of the corresponding target clock image.

As described above, the clock drawing test evaluation apparatus and method according to embodiments of the present disclosure can rapidly and consistently evaluate whether user exhibits a decline in cognitive function by analyzing a target clock image drawn by the user in real time.

The clock drawing test evaluation apparatus and method according to an embodiment of the present disclosure may enable the prevention and early detection of dementia and provide an environment in which a user can easily perform a clock drawing test and receive immediate feedback, thereby allowing continuous management of the user's cognitive state.

The clock drawing test evaluation apparatus and method according to an embodiment of the present disclosure can reduce variability in scoring caused by subjective expert judgment based on Artificial Intelligence (AI), and enable efficient handling even in situations requiring large-scale tests.

The term “unit” used in the present specification may include a unit implemented in hardware, software, or firmware, and may be interchangeably used with terms such as logic, logic block, component, or circuit. The term “unit” may be a component configured as an integrated body, or may be a minimum unit of the component, or a part thereof, that performs one or more functions. According to an embodiment, the term “unit” may be implemented in the form of an Application-Specific Integrated Circuit (ASIC).

The clock drawing test evaluation apparatus and method according to an aspect of the present disclosure can rapidly and consistently evaluate whether a user exhibits a decline in cognitive function by analyzing a clock image drawn by the user in real time.

The clock drawing test evaluation apparatus and method according to another aspect of the present disclosure may enable the prevention and early detection of dementia and provide an environment in which a user can easily perform a clock drawing test and receive immediate feedback, thereby allowing continuous management of the user's cognitive state.

The clock drawing test evaluation apparatus and method according to a further aspect of the present disclosure can reduce variability in scoring caused by subjective expert judgment based on Artificial Intelligence (AI), and enable efficient handling even in situations requiring large-scale tests.

The present disclosure has been described with reference to embodiments depicted in the drawings, but this is merely exemplary, and those skilled in the art will understand that various modifications and other equivalent embodiments are possible therefrom. Therefore, the technical scope of the present disclosure should be defined by the accompanying claims.

What is claimed is:

1. A clock drawing test evaluation apparatus, comprising:
a processor; and
a memory configured to store an instruction that is executed by the processor,
wherein the processor is configured to convert sketch data of a target clock image that is a subject of clock drawing test into a stroke-unit image including time information, extract feature vectors of the stroke-unit image from the stroke-unit image through a learning model, and then evaluate the clock drawing test based on the feature vectors of the stroke-unit image.

2. The clock drawing test evaluation apparatus of claim 1, wherein the processor is configured to assign a stroke order and range, a drawing time of the target clock image, and a time required to draw each stroke to three RGB channels, respectively.

3. The clock drawing test evaluation apparatus of claim 2, wherein the processor is configured to designate a color according to an order of strokes and normalize a range of strokes to assign a normalized stroke range to an R channel, normalize the drawing time of the target clock image to assign the normalized drawing time to a G channel, normalize a ratio of a time required to draw each stroke to a time required to complete an entire clock image to assign the normalized ratio to a B channel.

4. The clock drawing test evaluation apparatus of claim 1, wherein the learning model extracts feature vectors of training clock images and learns a distance proportional to a difference between scores of the training clock images based on the feature vectors of the training clock images.

5. The clock drawing test evaluation apparatus of claim 4, wherein:
the learning model learns the feature vectors of the training clock images through a Euclidean-distance-based loss function, and
the loss function reflects a distance margin in proportion to the difference between the scores of the training clock images.

6. The clock drawing test evaluation apparatus of claim 1, wherein the processor is configured to extract a score of the target clock image through a comparison of a similarity between the feature vectors of the stroke-unit image and feature vectors previously stored in a database unit.

7. The clock drawing test evaluation apparatus of claim 6, wherein the processor is configured to determine a score of any one feature vector having a highest similarity to the feature vectors of the stroke-unit image, among the feature vectors stored in the database unit, to be the score of the target clock image.

8. A clock drawing test evaluation method, comprising:
converting, by a processor, sketch data of a target clock image that is a subject of clock drawing test into a stroke-unit image including time information; and
extracting, by a processor, feature vectors of the stroke-unit image from the stroke-unit image through a learning model, and then evaluating the clock drawing test based on the feature vectors of the stroke-unit image.

9. The clock drawing test evaluation method of claim 8, wherein, in converting into the stroke-unit image, the processor is configured to assign a stroke order and range, a drawing time of the target clock image, and a time required to draw each stroke to three RGB channels, respectively.

10. The clock drawing test evaluation method of claim 9, wherein, in converting into the stroke-unit image, the processor is configured to designate a color according to an order of strokes and normalize a range of strokes to assign a normalized stroke range to an R channel, normalize the drawing time of the target clock image to assign the normalized drawing time to a G channel, normalize a ratio of a time required to draw each stroke to a time required to complete an entire clock image to assign the normalized ratio to a B channel.

11. The clock drawing test evaluation method of claim 8, wherein the learning model extracts feature vectors of training clock images and learns a distance proportional to a difference between scores of the training clock images based on the feature vectors of the training clock images.

12. The clock drawing test evaluation method of claim 11, wherein:
the learning model learns the feature vectors of the training clock images through a Euclidean-distance-based loss function, and
the loss function reflects a distance margin in proportion to the difference between the scores of the training clock images.

13. The clock drawing test evaluation method of claim 8, wherein, in evaluating the clock drawing test, the processor is configured to extract a score of the target clock image through a comparison of a similarity between the feature vectors of the stroke-unit image and feature vectors previously stored in a database unit.

14. The clock drawing test evaluation method of claim 13, wherein, in evaluating the clock drawing test, the processor is configured to determine a score of any one feature vector having a highest similarity to the feature vectors of the stroke-unit image, among the feature vectors stored in the database unit, to be the score of the target clock image.
